SEQUENCE LISTING (1 sequence)

SEQ ID NO: 1 - protein, 378 aa, synthetic construct
DVQLVESGGGLVQPGGSLRLSCAASGRTFSSYVVGWFRQAPGKEREFIGAISGSGESIYYAVSEKGRFTISRDNSKNTLYLQMNSLRPEDTAVYYCTADQEFGYLRFGRSEYWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGRTYDAMGWLRQAPGKEREFVAAISGSGDDTYYADSVKGRFTISRDNSKNTLYLQMNSLRPEDTAVYYCATRRGLYYVWDANDYENWGQGTLVTVSS

(19) United States
(12) Patent Application Publication
(10) Pub. No.: US 2025/0042988 A1
(43) Pub. Date: Feb. 6, 2025

(54) METHODS OF ACHIEVING SAFE AND SUSTAINED CONTROL OF IL-17-DEPENDENT CONDITIONS IN SUBJECTS RESPONSIVE TO TREATMENT WITH AN ANTI-IL 17A/F NANOBODY

(71) Applicant: MOONLAKE IMMUNOTHERAPEUTICS AG, Zug (CH)

(72) Inventor: Kristian REICH, Hamburg (DE)

(73) Assignee: MOONLAKE IMMUNOTHERAPEUTICS AG, Zug (CH)

(21) Appl. No.: 18/921,679

(22) Filed: Oct. 21, 2024

Related U.S. Application Data
(63) Continuation of application No. PCT/IB2023/054122, filed on Apr. 21, 2023.
(60) Provisional application No. 63/333,920, filed on Apr. 22, 2022.

(51) Int. Cl. C07K 16/24; A61P 17/06

(52) U.S. Cl. CPC C07K 16/244; A61P 17/06; C07K 2317/33; C07K 2317/569

(57) ABSTRACT

Disclosed herein are methods of treating psoriasis and other IL-17-dependent conditions with an anti-interleukin 17A/F nanobody for approximately 24 weeks, followed by withdrawal of treatment in responding patients with the anti-interleukin 17A/F nanobody for a period of at least 4-20 weeks. Disease modification may be achieved by no later than week 24 with the anti-interleukin 17A/F nanobody such that responders may demonstrate sustained normalization of peripheral and/or cutaneous biomarkers such as IL-17A, IL-17F, and/or IL-22. Treatment methods as contemplated herein which have been paused may be re-started based on the subject's condition. Treatment beyond 24 weeks also achieves unexpectedly high efficacy in responders who have not quite reached full skin clearance in the initial treatment period.

### CLAIM OF PRIORITY

The present application claims priority to U.S. Provisional Application No. 63/333,920, filed Apr. 22, 2022, which application is incorporated by reference herein in its entirety.

### FIELD OF INVENTION

The invention relates to the field of medical treatment of inflammatory conditions such as psoriasis and other IL-17-dependent conditions as well as biologics for use in such treatment.

### BACKGROUND

Optimal treatment of IL-17-dependent chronic inflammatory conditions such as psoriasis not only reduces disease activity during therapy, but also provides safe, long-term maintenance of control in responding patients after treatment withdrawal, sometimes referred to as “disease modification”. Maintenance of disease improvement has been observed many months following withdrawal of an IL-23 inhibitor in responding patients (Blauvelt et al., JAMA Dermatol 2020; 156:649-58). Models hypothesizing the perceived strength of IL-23 inhibitors in providing drug-free disease control focus on the upstream position of IL-23 in the psoriatic disease cascade and its role in inducing IL-17A production in IL-23-responsive T-cell subsets (Hawkes et al., J Immnol 2018; 201:1605-13).

The pro-inflammatory cytokine IL-17F is upregulated in psoriasis and other IL-17-dependent chronic inflammatory conditions, with accumulating evidence supporting its preferential production by yδ T cells, group 3 innate lymphoid cells and mucosa-associated invariate T cells (Hinks T S C and Zhang X W. Front Immunol 2020; 11:1014; Cole S, et al. Front Immunol 2020; 11:585134; Domingues R G and Hepworth M R. Front Immunol 2020; 11:116; Provine N M, et al. Science. 2021; 371:521-26). There is also strong evidence that IL-17F production in these cells is at least partially independent of IL-23 (Cole et al., Front Immunol 2020; 11:585134).

IL-17A is also upregulated in psoriasis, such that both IL-17A and IL-17F are biologically active as dimers (IL-17A/A, IL-17A/F, and IL-17F/F) that signal through a receptor composed of two IL-17 receptor A chains (IL-17RA) and one IL-17 receptor C chain.

Biomarkers measured in the peripheral blood and/or affected tissue will a) help to predict the disease course and b) response to targeted treatment of IL-17-dependent chronic inflammatory conditions such as psoriasis. These biomarkers reflect disease-underlying pathways as well as the targets of IL-17 inhibiting therapies used for the short- and long-term control of inflammation. For example, IL-17 induces in keratinocytes the production of neutrophil-chemoattractive factors such as CXCL8 (formerly IL-8) that enhance and perpetuate inflammation in diseases such as psoriasis and hidradenitis suppurativa (Narla S, et al. Br J Dermatol. 2021 June; 184(6):1004-1013) characterized by the influx of neutrophils to diseased tissue. Biomarkers relevant to optimize disease management during treatment with IL-17 inhibitors will be shared between dermatological and rheumatological conditions responsive to IL-17 blockade. For example, IL-22 plays a role as predictive marker not only in IL-17-dependent skin but also joint conditions such as psoriatic arthritis (Miyagawa I, et al. Arthritis Res Ther. 2022 Apr. 15; 24(1):86. doi: 10.1186/s13075-022-02771-4). Biomarkers related to the production of antimicrobial peptides such as the protein encoded by DEFB4A will also c) identify patients at an increased risk to develop side effects during treatment with IL-17 inhibiting drugs.

Monoclonal antibodies that target IL-17 are known and have been utilized as treatments for plaque-type psoriasis (Gordon et al., N Engl J Med 2016; 375:345-56; Langley et al., N Engl J Med 2014; 371:326-28). However, canonical antibodies have certain shortcomings including their large size (approximately 150 kDa) and limited tissue penetration, relative instability, as well as their relative expense in manufacturing.

Nanobodies are a class of therapeutic proteins based on single-domain, camelid, heavy-chain-only antibodies which can overcome such limitations by combining the advantages of small proteins with the benefits of monoclonal antibody properties. Nanobodies are not glycosylated and therefore allow for alternative expression hosts with considerably higher volumetric yields than monoclonal antibodies. Also, compared with conventional antibodies, nanobodies exhibit a high stability in terms of fragmentation, aggregation propensity, and stress susceptibility (Konning et al., Curr Opin Struct Biol 2017; 45:10-16).

Sonelokimab (also known as M1095) is a trivalent nanobody comprised of monovalent camelid-derived (i.e., from the Camelidae family of mammals, such as camels, llamas and alpacas) nanobodies specific to human interleukin (IL)-17A, IL-17F, and human serum albumin.

Long term treatment with immune-regulating biologics is not only extremely costly but includes risk to the patient of infection and other health issues. Conventional antibody treatment of 17-dependent conditions such as psoriasis often results in administration of doses higher than necessary and for periods of time longer than necessary. There remains a need for treatment methods capable of achieving sustained control of such conditions that avoid these disadvantages.

### Summary of the Invention

Disclosed herein is a method of treating an IL-17-dependent condition comprising administering a nanobody which specifically binds to an IL17A/A homodimer, an IL17-A/F heterodimer, and/or an IL-17F/F homodimer to a subject in need thereof at a dose of 30-240 mg every two to four weeks for up to 24 weeks, followed by withdrawal of treatment with the nanobody for responding subjects. In an embodiment, the administering is performed subcutaneously. In an embodiment, the withdrawal of treatment with the nanobody is for a period of 4 weeks or more, preferably for a period of 10 weeks or more, even more preferably for a period of 20 weeks or more (e.g., 8, 10, 12, 14, 16, 18, or 20 weeks).

For example, methods as disclosed herein include methods for treating an IL-17-dependent dermatological condition. In certain embodiments, the method may be for treating psoriasis (including but not limited to plaque psoriasis, moderate-to-severe plaque psoriasis, pustular psoriasis, generalized pustular psoriasis, palmo-plantar psoriasis, scalp psoriasis, guttate psoriasis, erythrodermic psoriasis, inversive psoriasis), atopic dermatitis, discoid lupus erythematosus, alopecia areata, autoimmune urticaria, bullous pemphigoid, dermatitis herpetiformis, hidradenitis suppurativa, linear IgA dermatosis, morphea, pemphigus vulgaris, or pyoderma gangrenosum.

In certain embodiments, the method may be for treating psoriatic arthritis, axial spondyloarthritis including ankylosing spondylitis, systemic lupus erythematosus, rheumatoid arthritis, vasculitis, Sjogren's syndrome, juvenile idiopathic arthritis, granulomatosis, Behcet's disease, antiphospholipid syndrome, giant cell arteritis, scleroderma, polyarteritis nodosa, or Takayasu disease.

In an embodiment, the methods as above may comprise administering the nanobody at a dose of 30, 60, 120, or 240 mg every two to four weeks for up to 24 weeks.

In an embodiment, the methods as above include such methods, wherein the dose is increased between week 2 and week 24 if the subject has an IGA score of more than 1.

In an embodiment, the nanobody comprises sonelokimab.

In an embodiment, methods as above comprises a method for treating psoriasis and which achieves full skin clearance in the subject by as early as week 4 to week 8.

Also disclosed herein is a method of disease modification comprising administering 30-240 mg of a nanobody to a subject suffering from IL-17-dependent condition every two weeks, wherein the nanobody specifically binds to an IL17A/A homodimer, an IL17-A/F heterodimer, and/or an IL-17F/F homodimer; and the treatment is for a period of no less than 4 weeks, optionally no less than 8 weeks, and preferably for a period of no longer than 24 weeks. In an embodiment, the method of disease modification comprises administering the nanobody every two weeks until week 12, and then every four weeks until week 24. In an embodiment, the method of disease modification may be a method in which administration of the nanobody is paused or permanently stopped at week 24.

In an embodiment, the disease modification method achieves normalization of peripheral IL-17A, IL-17F, CCL20, CXCl1, DEFB4A, CXCL8, LCN2, CAMP, KRT16, IL-13, IL-23, IL-31 and/or IL-22 in the subject by no later than week 24.

In an embodiment, the disease modification method achieves normalization of cutaneous IL-17A, IL-17F, CCL20, CXCl1, DEFB4A, CXCL8, LCN2, CAMP, KRT16, IL-13, IL-23, IL-31 and/or IL-22 in the subject by no later than week 24.

In an embodiment, the normalization is sustained for at least 4, 8, 12, 16, and/or 20 additional weeks after treatment has been paused or stopped.

Also contemplated herein are such disease modification methods which comprise: administering the nanobody for a total of at least 4 weeks and preferably for a total of no more than 24 weeks, pausing the treatment for a period of more than two weeks, and then re-initiating treatment if one or more symptoms of the IL-17-dependent condition recur. In an embodiment, administration may be re-started when the subject has an IGA score of 1 or more. In an embodiment, re-initiating treatment may comprise administering 30-240 mg of the nanobody every two weeks.

In certain embodiments, in the disease modification methods disclosed herein, the IL-17-dependent condition is a dermatological condition.

For example, the method may be a method for treating psoriasis (including but not limited to plaque psoriasis, moderate-to-severe plaque psoriasis, pustular psoriasis, generalized pustular psoriasis, palmo-plantar psoriasis, scalp psoriasis, guttate psoriasis, erythrodermic psoriasis, inversive psoriasis), atopic dermatitis, discoid lupus erythematosus, alopecia areata, autoimmune urticaria, bullous pemphigoid, dermatitis herpetiformis, hidradenitis suppurativa, linear IgA dermatosis, morphea, pemphigus vulgaris, or pyoderma gangrenosum.

In an embodiment, in disease modification methods as above, the nanobody may comprise sonelokimab.

In an embodiment, the disease modification includes methods in which the administering is performed subcutaneously.

Also disclosed herein is a method of treating an IL-17-dependent dermatological condition comprising administering a nanobody which specifically binds to an IL17A/A homodimer, an IL17-A/F heterodimer, and/or an IL-17F/F homodimer to a subject in need thereof at a dose of 30-240 mg every two to four weeks continuously for more than 24 weeks to achieve full skin clearance. In an embodiment, such a method achieves an complete clearance of the disease in 40% or more, preferably 50% or more, of subjects still exhibiting symptoms of the IL-17-dependent dermatological condition at week 24 of treatment.

Also disclosed herein is use of a medicament comprising a nanobody which specifically binds to an IL17A/A homodimer, an IL-17A/F heterodimer, and/or an IL-17F/F homodimer to a subject in need thereof at a dose of 30-240 mg every two to four weeks for up to 24 weeks, followed by withdrawal of the treatment with the medicament comprising the nanobody for a subject responsive to the treatment; wherein the subject is a human or non-human animal.

In an embodiment, the subject in need thereof has a dermatological condition.

In an embodiment, contemplated herein is such a use in which the subject in need thereof has or is at risk of having psoriasis (including but not limited to plaque psoriasis, moderate-to-severe plaque psoriasis, pustular psoriasis, generalized pustular psoriasis, palmo-plantar psoriasis, scalp psoriasis, guttate psoriasis, erythrodermic psoriasis, inversive psoriasis), atopic dermatitis, discoid lupus erythematosus, alopecia areata, autoimmune urticaria, bullous pemphigoid, dermatitis herpetiformis, hidradenitis suppurativa, linear IgA dermatosis, morphea, pemphigus vulgaris, or pyoderma gangrenosum.

In an embodiment also disclosed is such a use in which the subject in need thereof has or is at risk of having psoriatic arthritis, axial spondyloarthritis including ankylosing spondylitis, systemic lupus erythematosus, rheumatoid arthritis, vasculitis, Sjogren's syndrome, juvenile idiopathic arthritis, granulomatosis, Behcet's disease, antiphospholipid syndrome, giant cell arteritis, scleroderma, polyarteritis nodosa, or Takayasu disease.

In an embodiment, the subject may have psoriasis, hidradenitis suppurativa, psoriatic arthritis, or axial spondyloarthritis, and treatment may be withdrawn when the subject reaches a certain clinical threshold, e.g., PASI score of 75-100 (psoriasis), IGA score of 0 or 1 (psoriasis), a Hidradenitis Suppurativa Clinical Response (HiSCR) score of 75-90 (hidradenitis suppurativa), an American College of Rheumatology criteria (ACR) score of 50 to 70 (psoriatic arthritis), or an Assessment in SpondyloArthritis International Society (ASAS) score of 40 or more (axial spondyloarthritis).

In an embodiment, the subject responsive to treatment may have a PASI score of 75-100, preferably a PASI score of 90-100, most preferably a PASI score of 100 after a period of 4 weeks or more of treatment, after a period of 8 weeks or more of treatment, after a period of 10 weeks or more of treatment, after a period of 12 weeks or more of treatment, after a period of 14 weeks or more of treatment, after a period of 16 weeks or more of treatment, after a period of 18 weeks or more of treatment, after a period of 20 weeks or more of treatment, or after a period of 24 weeks. In such an embodiment, the subject responsive to treatment has or is at risk of having psoriasis and treatment may be withdrawn after a period 4 weeks or more, after a period of 8 weeks or more, after a period of 10 weeks or more, after a period of 12 weeks or more, after a period of 14 weeks or more, after a period of 16 weeks or more, after a period of 18 weeks or more, after a period of 20 weeks or more, or after a period of 24 weeks of treatment.

In an embodiment, the subject responsive to treatment may have an IGA score of 0 or 1, preferably an IGA of 0, after a period of 4 weeks or more of treatment, after a period of 8 weeks or more of treatment, after a period of 10 weeks or more of treatment, after a period of 12 weeks or more of treatment, after a period of 14 weeks or more of treatment, after a period of 16 weeks or more of treatment, after a period of 18 weeks or more of treatment, after a period of 20 weeks or more of treatment, or after a period of 24 weeks of treatment. In such an embodiment, the subject responsive to treatment has or is at risk of having psoriasis and in which treatment is withdrawn after a period of 4 weeks or more of treatment, after a period of 8 weeks or more, after a period of 10 weeks or more, after a period of 12 weeks or more, after a period of 14 weeks or more, after a period of 16 weeks or more, after a period of 18 weeks or more, after a period of 20 weeks or more, or after a period of 24 weeks of treatment.

In an embodiment, the subject responsive to treatment has an HiSCR score of 75 to 90, preferably an HiSCR score of 90, after a period of 4 weeks or more of treatment, after a period of 8 weeks or more of treatment, after a period of 10 weeks or more of treatment, after a period of 12 weeks or more of treatment, after a period of 14 weeks or more of treatment, after a period of 16 weeks or more of treatment, after a period of 18 weeks or more of treatment, after a period of 20 weeks or more of treatment, or after a period of 24 weeks of treatment. In such an embodiment, the subject responsive to treatment has or is at risk of having hidradenitis suppurativa and in which treatment is withdrawn after a period of 4 weeks or more, after a period of 8 weeks or more, after a period of 10 weeks or more, after a period of 12 weeks or more, after a period of 14 weeks or more, after a period of 16 weeks or more, after a period of 18 weeks or more, after a period of 20 weeks or more, or after a period of 24 weeks of treatment.

In an embodiment, the subject responsive to treatment has an ACR score of 50 to 70, preferably an ACR score of 70, after a period of 4 weeks or more of treatment, after a period of 8 weeks or more of treatment, after a period of 10 weeks or more of treatment, after a period of 12 weeks or more of treatment, after a period of 14 weeks or more of treatment, after a period of 16 weeks or more of treatment, after a period of 18 weeks or more of treatment, after a period of 20 weeks or more of treatment, or after a period of 24 weeks of treatment. In such an embodiment, the subject responsive to treatment has or is at risk of having psoriatic arthritis and in which treatment is withdrawn after a period of 4 weeks or more, after a period of 8 weeks or more, after a period of 10 weeks or more, after a period of 12 weeks or more, after a period of 14 weeks or more, after a period of 16 weeks or more, after a period of 18 weeks or more, after a period of 20 weeks or more, or after a period of 24 weeks of treatment.

In an embodiment, the subject responsive to treatment has an ASAS score of 40 or more after a period of 4 weeks or more of treatment, after a period of 8 weeks or more of treatment, after a period of 10 weeks or more of treatment, after a period of 12 weeks or more of treatment, after a period of 14 weeks or more of treatment, after a period of 16 weeks or more of treatment, after a period of 18 weeks or more of treatment, after a period of 20 weeks or more of treatment, or after a period of 24 weeks of treatment. In such an embodiment, the subject responsive to treatment has or is at risk of having axial spondyloarthritis and in which treatment is withdrawn after a period of 4 weeks or more, after a period of 8 weeks or more, after a period of 10 weeks or more, after a period of 12 weeks or more, after a period of 14 weeks or more, after a period of 16 weeks or more, after a period of 18 weeks or more, after a period of 20 weeks or more, or after a period of 24 weeks of treatment.

### BRIEF DESCRIPTION OF THE DRAWINGS

FIG. 1. Study design of placebo-controlled phase 2b study with sonelokimab in psoriasis. The IL-17A inhibiting antibody secukinumab served as active reference.

FIG. 2. Investigator's Global Assessment (IGA) and Psoriasis Area and Severity Index (PASI) response rates in the intention-to-treat population from baseline to week 24 in the various treatment groups.

FIG. 3. PASI 90 (A) and PASI 100 (B) response rates in participants randomized at study baseline to receive 120 mg of sonelokimab (normal and augmented load) who achieved clear skin at week 24 and had sonelokimab withheld and re-started upon disease re-occurrence during weeks 24-48 (blue/top lines) or had remaining psoriasis lesions at week 24 and received sonelokimab continuously every 4 weeks during weeks 24-28 (red/bottom lines).

FIG. 4. Drug-free remission and treatment re-initiation among patients treated with sonelokimab 120 mg (augmented load) achieving complete skin clearance (IGA=0) at week 24. Sonelokimab was withdrawn in all of these patients at week 24 and restarted only if disease control was lost (IGA≥1; visit interval=every 4 weeks). Percentages in bold indicate patients with complete clearance at each visit with no re-treatment. Complete clearance was maintained until week 44 without treatment re-initiation in 20% (n=5/25) of patients. Percentages of patients restarted at each visit are given next to the blue/curved arrows; *n indicates the number of restarted patients again achieving IGA=0. Of 20 patients overall re-started on sonelokimab 80% achieved again complete clearance. The green arrowhead indicates the time of the last sonelokimab injection before treatment withdrawal. The orange arrowhead indicates approximately 5× half-life of sonelokimab after the last dose.

FIG. 5. Percentage of patients with skin clearance (IGA=0) over time among patients achieving skin clearance at week 24. In patients receiving sonelokimab (120 mg, augmented load) the drug was stopped and only re-started upon re-occurrence of psoriasis (blue dotted line). In patients receiving the IL-17A inhibiting antibody secukinumab (approved dose) treatment was continuously with no withdrawal (orange solid line). Bars represent the percentage of patients receiving injections of sonelokimab (light blue bars) or secukinumab (light orange bars) at each visit (every 4 weeks). Similar rates of skin clearance were seen for both drugs at week 48 with patients in the sonelokimab group receiving only 50% of the overall monthly injections compared with patients in the secukinumab group.

FIG. 6. Skin clearance rates over time among patients with active disease (IGA≥1) at week 24 receiving continuous treatment with secukinuamb (solid orange line) or sonelokimab (solid blue line). Complete skin clearance at week 48 was achieved in 50.0% of the patients treated with sonelokimab compared to 30.0% of the patients treated with secukinumab (P=0.166).

FIG. 7. Demographics and baseline disease in the intention-to-treat population. Data are mean (SD) or n (%), unless otherwise specified.

FIG. 8. IGA and PASI responses at weeks 12 and 24 in the intention-to-treat population. Data are n (%; 95% CI). For PASI scores, any missing response was imputed as a non-response. PASI<3=a numerical PASI score of less than 3. PASI 75=at least 75% improvement from baseline. PASI 90=at least 90% improvement from baseline. PASI 100=100% improvement from baseline. *p<0.0001 for all groups versus the placebogroup, unless otherwise indicated. †A PASI<3 response was not a prespecified endpoint. ‡p=0.0016. § p=0.0003. ¶Included participants who received sonelokimab 120 mg normal load once every 4 weeks from week 12 in the placebo group; sonelokimab 30 mg and escalated sonelokimab 120 mg once every 4 weeks after week 12 in the sonelokimab 30 mg normal load group; sonelokimab 60 mg and escalated sonelokimab 120 mg once every 4 weeks after week 12 in the sonelokimab 60 mg normal load group; sonelokimab 120 mg once every 8 weeks after week 12 in the sonelokimab 120 mg normal load group; sonelokimab 120 mg once every 4 weeks after week 12 in the sonelokimab 120 mg augmented load group; and secukinumab 300 mg once every 4 weeks after week 12 in the secukinumab 300 mg group.

FIG. 9. Summary of safety and tolerability results at weeks 0-12 and 12-52 in the safety analysis population. Data are n (%). *See Supplement in Papp et al., Lancet 2021; 397:1564-75, which is incorporated by reference herein in its entirety, for information on specific events. †During weeks 0-12, common treatment-emergent adverse events were considered as those occurring in 5% or more of participants in any of the sonelokimab-containing groups; during weeks 12-52, common treatment-emergent adverse events were considered as those occurring in 3% of all participants in the all sonelokimab-containing groups combined. ‡Events under preferred term of oral candidiasis for weeks 12-24; see adverse events of special interest for consolidated Candida assessment. § Includes infections, injection site reactions, liver function test abnormalities, cerebrocardiovascular events, cytopenia, allergic or hypersensitivity reactions, malignancies, depression, and inflammatory bowel disease. ¶Post-hoc consolidation of adverse event terms to assess oral, oesophageal, and vaginal candidiasis (participants with oral candidiasis, Candida infection, oesophageal candidiasis, oropharyngeal candidiasis, or vulvovaginal candidiasis). **Includes myocardialinfarction, cerebrovascular accident, or cardiovascular death.

FIG. 10. Patient Disposition.

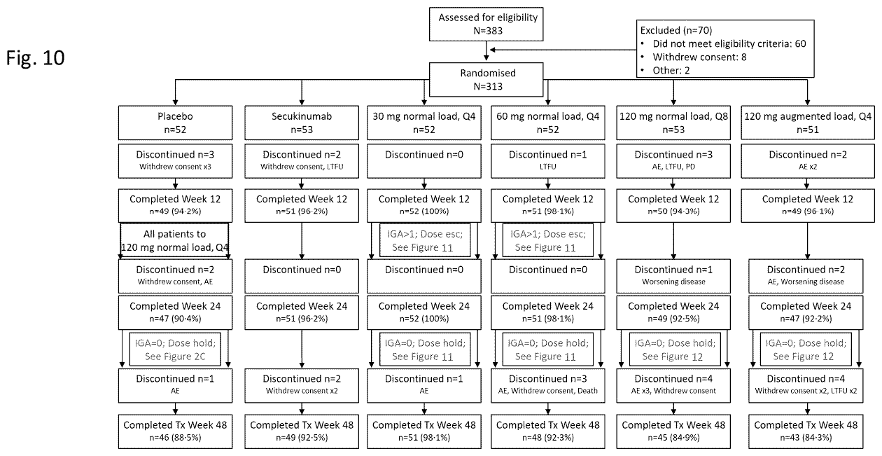

FIG. 11. Patient Disposition.

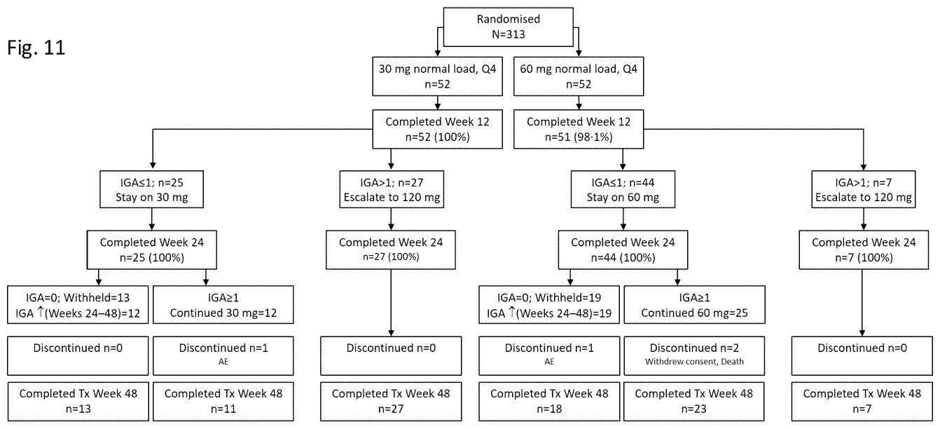

FIG. 12. Patient Disposition.

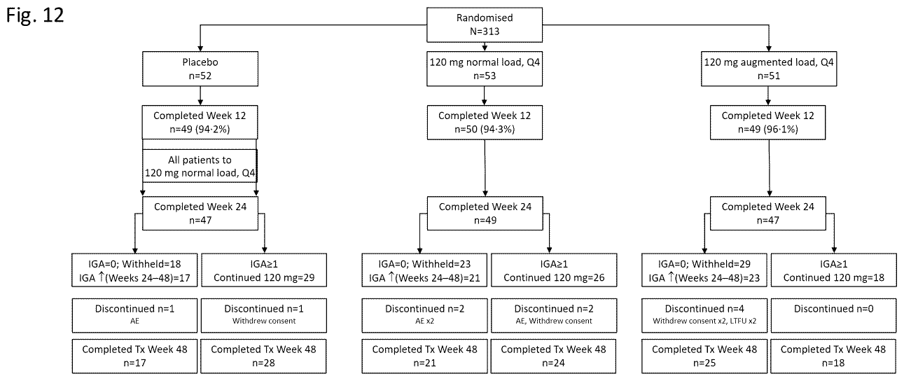

FIG. 13. IGA Score Category Shift From Baseline to Week 12 (ITT Population).

FIG. 14. DLQI 0/1 Response Rates (NRI) at Week 12 and 24 (ITT Population).

FIG. 15. Serious Adverse Events by Preferred Term (Weeks 0-52).

### DETAILED DESCRIPTION OF THE INVENTION

The particulars shown herein are by way of example and for purposes of illustrative discussion of the various embodiments only and are presented in the cause of providing what is believed to be the most useful and readily understood description of the principles and conceptual aspects of the methods and compositions described herein. In this regard, no attempt is made to show more detail than is necessary for a fundamental understanding, the description making apparent to those skilled in the art how the several forms may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope to those skilled in the art.

Unless otherwise defined, all technical and scientific terms used herein have the same meaning as commonly understood by one of ordinary skill in the art to which this invention belongs. The terminology used in the description herein is for describing particular embodiments only and is not intended to be limiting. As used in the description and the appended claims, the singular forms “a,” “an,” and “the” are intended to include the plural forms as well, unless the context clearly indicates otherwise. All publications, patent applications, patents, and other references mentioned herein are expressly incorporated by reference in their entirety.

Unless indicated to the contrary, the numerical parameters set forth in the following specification and attached claims are approximations that may vary depending upon the desired properties sought to be obtained and thus may be modified by the term “about”. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein. Applicant also contemplates ranges derived from data points and express ranges disclosed herein.

### Nanobodies

Nanobodies are a novel class of proprietary therapeutic proteins based on the smallest functional fragments of heavy chain only antibodies (Vhh) that are naturally found in the Camelidae family. Nanobodies bind their target with the specificity and affinity of a traditional therapeutic antibody, but a single Vhh has a size of only around 15 kDa which is approximately 10 times smaller than a traditional antibody. The smaller size comes with a number of advantages; a) Several Vhhs can be linked to more easily form multivalent molecules that overcome the limitations of traditional mono-specific antibodies in complex disease driven by multiple cytokines or in the passive immunization against viral diseases (Koenig P-A, et al. Science 2021; 371(6530):eabe6230). b) The smaller size of nanobodies still allows the binding to and inhibition of mutated growth hormone receptors on tumor cells resistant to traditional antibodies with the same specificity (Tintelnot J, et al. Mol Cancer Ther 2019; 18:823-33). c) There is solid evidence that the smaller size of nanobodies allows a better tissue penetration and specific enrichment at sites of malignant or inflammatory disease compared to traditional antibodies (Krüwel T, et al. Sci Rep 2016; 6:21834). The latter phenomenon is further enhanced with nanobodies carrying an albumin-binding part such as sonelokimab enabling preferential accumulation within inflammatory edema, for example swollen joints (Coppieters K, et al. Arthritis Rheum 2006; 54:1856-66). Sonelokimab is a trivalent single chain monoclonal nanobody, consisting of three sequence-optimized Vhhs (each derived from a heavy-chain-only llama antibody) that contain the unique structural and functional properties of naturally occurring heavy-chain-only antibodies. The N-terminal moiety binds to IL-17F and the C-terminal moiety to IL-17A and IL-17F allowing inhibition of the biologically relevant IL-17A/A, IL-17A/F and IL-17F/F dimers. The central moiety binds to serum albumin. The subunits are fused head-to-tail with a 9-amino acid glycine/serine linker resulting in a total predicted molecular weight of around 40 kDa. The calculated 4 to 6 times higher drug tissue penetration of sonelokimab compared to traditional antibodies (150 kDa) (Li Z, et al. mAbs 2016; 8:1:113-119) is an important consideration in the treatment especially of inflammatory diseases such as hidradenitis suppurativa where disease presentation is often characterized by inflammatory lesions surrounded by scar tissue and deep inflammatory dermal morphologies such as tunnels.

Nanobodies for use herein include nanobodies as disclosed in U.S. Pat. No. 10,017,568, which document is incorporated by reference herein in its entirety.

Nanobodies for use herein, including SEQ ID NO:1, and variants thereof are contemplated. For example, a nanobody having a sequence homologous to the amino acid sequence represented by SEQ ID NO: 1 is included, and may be a protein having the same amino acid sequence as the amino acid sequence represented by SEQ ID NO: 1 except that one or several amino acids are deleted, substituted, inserted, and/or added. In the case of substitution, insertion, or addition, conservative mutations resulting from conservative substitution, insertion, or addition of one or several amino acids are possible.

“One or several amino acids” herein means 1 to 50, preferably 1 to 20, more preferably 1 to 10, still more preferably 1 to 5 or 1 to 3, amino acids.

Moreover, a protein having an amino acid sequence homologous to the amino acid sequence represented by SEQ ID NO: 1 includes a protein having an amino acid sequence with an identity of not less than 70% to the amino acid sequence represented by SEQ ID NO: 1, in its full-length form. The protein includes a protein having an amino acid sequence with an identity of preferably not less than 80%, more preferably not less than 90%, and still more preferably not less than 95%, 96%, 97%, 98%, or 99% to the above-described amino acid sequence in its full-length form.

“Sequence identity” may refer, in nucleotide sequences or amino acid sequences, to the percentage of identical nucleotides or amino acids shared between two sequences, which percentage is determined by aligning those two sequences in an optimal pairwise alignment, optionally by using a conventional or commercially available algorithm.

### Disease Modification

Disease modification is commonly used in the context of therapeutic disease management to describe treatment effects that change the natural course of a disease. For example, the term disease-modifying anti-rheumatic drugs (DMARDs) is used to describe treatments that prevent the occurrence and/or progression of bone destruction typically characterizing the (untreated) course of diseases such as rheumatoid or psoriatic arthritis. More recently, disease modification has been discussed to underly the maintenance of disease control in responding patients with psoriasis after treatment withdrawal. For example, the prolonged disease control observed after the termination of treatment with antibodies inhibiting the cytokine IL-23 in patients with psoriasis, in whom the natural disease course would predict a rapid re-occurrence of the disease, has been classified to represent disease modification Eyerich K, et al. BMJ Open. 2021 Sep. 13; 11(9):e049822) and has been linked to the upstream role of IL-23 in the psoriatic disease cascade as well as specific molecular and cellular phenomena in previously affected skin such as the reduction of resident memory CD8+ T cells (Mehta H, et al. J Invest Dermatol. 2021 July; 141(7):1707-1718). An alternative definition of disease modification in the treatment of chronic inflammatory skin conditions has been described an increasing improvement of skin lesions with continuous treatment with parallel documentation of molecular and/or cellular normalization of affected tissue (Bieber T, et al. Allergy. 2012 August; 67(8):969-75). Such a definition may be applicable to disease such as atopic dermatitis and hidradenitis suppurativa in which complete skin clearance is not achievable with the currently existing therapies.

The more recent identification of other tissue resident immune cells such as innate lymphoid cells and yδ T cells that produce the pro-inflammatory cytokine IL-17F independent of IL-23 has raised the question whether inhibitors of IL-17, especially of IL-17F, may also show phenomena related to disease modification. This is particularly relevant in diseases in which IL-17F has been identified as a main driver of inflammation and/or inhibitors of IL-23 have demonstrated only limited or no relevant clinical effects such as hidradenitis suppurativa, psoriatic arthritis and ankylosing spondylitis.

### Administration and Dosing

Nanobodies for the prevention and/or treatment of the diseases and conditions mentioned herein will, depending on the disease or condition to be treated, the specific route of administration, and the individualized regime for the subject, be administered at a dose of 30 mg to 240 mg. Contemplated induction injection schemes include administration every 2 weeks until week 8 to 12; contemplated maintenance regimes include administration every 4 weeks to every 8 weeks. Early response to treatment may occur after 2 to 8 weeks; time to full response, i.e. skin clearance, may be up to 48 weeks; therefore, first time to withdrawal may be after 16 to 48 weeks.

Generally, for pharmaceutical use, the nanobodies of the invention may be formulated as a pharmaceutical preparation or compositions comprising the nanobody and at least one pharmaceutically acceptable carrier, diluent or excipient and/or adjuvant, and optionally one or more further pharmaceutically active polypeptides and/or compounds. By means of non-limiting examples, such a formulation may be in a form suitable for oral administration, for parenteral administration (such as by intravenous, intramuscular or subcutaneous injection or intravenous infusion, intraluminal, intra-arterial or intrathecal administration), for topical (i.e. transdermal or intradermal) administration, for administration by inhalation, by a skin patch, by an implant, by a suppository, etc. Such suitable administration forms—which may be solid, semi-solid or liquid, depending on the manner of administration—as well as methods and carriers for use in the preparation thereof, will be clear to one of skill in the art.

### Subjects

Patients with an increased susceptibility to disease modification induced by treatment with sonelokimab are likely patients with shorter disease duration, for example less than 6 months to 2 years; patients with early response of serum and/or tissue biomarkers, for example after 2 to 8 weeks; patients with early and high levels of clinical response, for example after 2 to 8 weeks, 50% to 90% improvement; and/or patients with high and stable levels of response, for example PASI 100 at week 12 and one or two consecutive visits 2 to 6 weeks apart.

Biomarkers to predict disease severity, disease course, response to treatment and/or susceptibility to side effects to inhibition of IL-17A and IL-17F may be based on measures a) of proteins in the peripheral blood and/or mRNA in circulating cells and/or analysis of surface markers and/or intracellular markers in peripheral cells by flow cytometry; and/or b) of proteins measured in tissue; levels of mRNA in tissue, bulk and/or single cell RNAseq of cells isolated from tissue. Useful biomarkers as contemplated herein include, but are not limited to, IL-17A, IL-17F, CCL20, CXCl1, DEFB4A, CXCL8, LCN2, CAMP, KRT16, IL-13, IL-23, IL-31 and/or IL-22.

### EXAMPLES

### Example 1

In a 48-week Phase 2b study in patients with moderate-to-severe plaque-type psoriasis (n=313), the IL-17A- and IL-17F-targeting nanobody sonelokimab was withdrawn at week 24 in patients achieving complete skin clearance (investigator global assessment [IGA]=0) and restarted if disease control was lost (IGA≥1). Sonelokimab comprises the following amino acid sequence: Asp Val Gln Leu Val Glu Ser Gly Gly Gly Leu Val Gln Pro Gly Gly Ser Leu Arg Leu Ser Cys Ala Ala Ser Gly Arg Thr Phe Ser Ser Tyr Val Val Gly Trp Phe Arg Gln Ala Pro Gly Lys Glu Arg Glu Phe Ile Gly Ala Ile Ser Gly Ser Gly Glu Ser Ile Tyr Tyr Ala Val Ser Glu Lys Gly Arg Phe Thr Ile Ser Arg Asp Asn Ser Lys Asn Thr Leu Tyr Leu Gln Met Asn Ser Leu Arg Pro Glu Asp Thr Ala Val Tyr Tyr Cys Thr Ala Asp Gln Glu Phe Gly Tyr Leu Arg Phe Gly Arg Ser Glu Tyr Trp Gly Gln Gly Thr Leu Val Thr Val Ser Ser Gly Gly Gly Gly Ser Gly Gly Gly Ser Glu Val Gln Leu Val Glu Ser Gly Gly Gly Leu Val Gln Pro Gly Asn Ser Leu Arg Leu Ser Cys Ala Ala Ser Gly Phe Thr Phe Ser Ser Phe Gly Met Ser Trp Val Arg Gln Ala Pro Gly Lys Gly Leu Glu Trp Val Ser Ser Ile Ser Gly Ser Gly Ser Asp Thr Leu Tyr Ala Asp Ser Val Lys Gly Arg Phe Thr Ile Ser Arg Asp Asn Ala Lys Thr Thr Leu Tyr Leu Gln Met Asn Ser Leu Arg Pro Glu Asp Thr Ala Val Tyr Tyr Cys Thr Ile Gly Gly Ser Leu Ser Arg Ser Ser Gln Gly Thr Leu Val Thr Val Ser Ser Gly Gly Gly Gly Ser Gly Gly Gly Ser Glu Val Gln Leu Val Glu Ser Gly Gly Gly Leu Val Gln Pro Gly Gly Ser Leu Arg Leu Ser Cys Ala Ala Ser Gly Arg Thr Tyr Asp Ala Met Gly Trp Leu Arg Gln Ala Pro Gly Lys Glu Arg Glu Phe Val Ala Ala Ile Ser Gly Ser Gly Asp Asp Thr Tyr Tyr Ala Asp Ser Val Lys Gly Arg Phe Thr Ile Ser Arg Asp Asn Ser Lys Asn Thr Leu Tyr Leu Gln Met Asn Ser Leu Arg Pro Glu Asp Thr Ala Val Tyr Tyr Cys Ala Thr Arg Arg Gly Leu Tyr Tyr Val Trp Asp Ala Asn Asp Tyr Glu Asn Trp Gly Gln Gly Thr Leu Val Thr Val Ser Ser (SEQ ID NO: 1).

### Participants

Adults aged 18-75 years, with stable, moderate to severe, plaque-type psoriasis (defined as an Investigator's Global Assessment [IGA] score of ≥3, a body surface area involvement of ≥10%, and a Psoriasis Area and Severity Index [PASI] score of ≥12) for more than 6 months before randomisation, who were candidates for systemic biologic therapy, and who did not have underlying medical conditions that put them at an unacceptable risk for receiving an immunomodulatory therapy, were eligible for this study. Patients previously treated with more than two biological therapies or any therapy targeting IL-17 were excluded. All patients provided written informed consent before screening.

### Randomization and Masking

Participants were enrolled by masked investigators and randomly assigned (1:1:1:1:1:1) to receive either placebo, sonelokimab 30 mg, sonelokimab 60 mg, sonelokimab 120 mg normal load, sonelokimab 120 mg augmented load, or secukinumab 300 mg. Randomisation was stratified by weight (≤90 kg or >90 kg) and previous use of biologics (previous use or no previous use), which resulted in four strata, with a permuted block size of six within each stratum. Randomisation was done at a study level via a centralised interactive response technology system, which provided blinded treatment kit numbers to the investigator. Study drug was prepared and administered at the site by a designated unmasked individual at the study site, who had no other involvement in the trial. Participants and all other site personnel were masked to therapy allocation throughout the study. The study sponsor was unmasked after all participants had completed week 24 treatment and the database was locked.

### Procedures

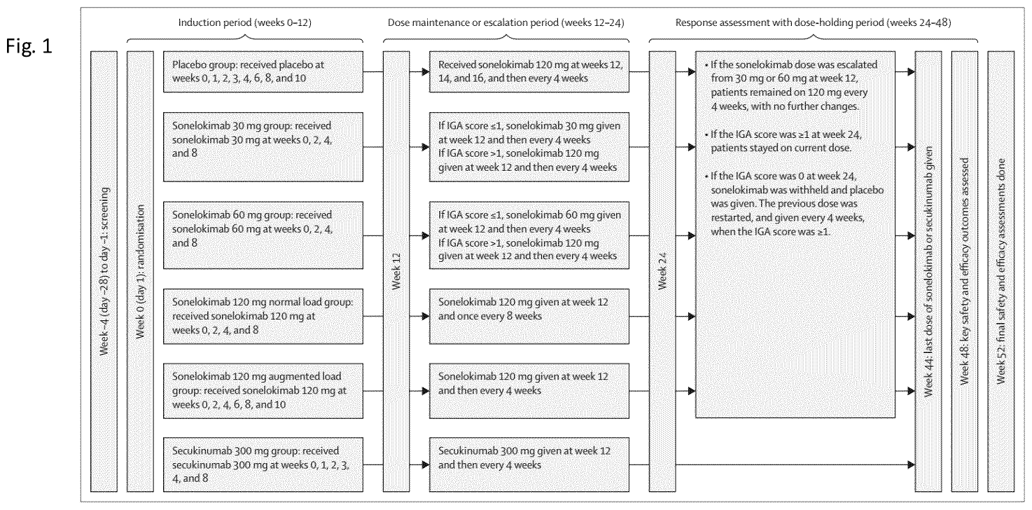

All participants underwent a 4-week screening period, a 12-week placebo-controlled induction period, a 12-week dose maintenance or escalation period (during which all participants were on active treatment), a 24-week response assessment or dose-holding period, and a final assessment in week 52 (FIG. 1). Use of bland emollients were allowed throughout the study period, apart from during the 24-h period before a clinic visit.

During the placebo-controlled induction period (weeks 0-12), participants received either placebo (at weeks 0, 1, 2, 3, 4, 6, 8, and 10), sonelokimab 30 mg, 60 mg, or 120 mg normal load (at weeks 0, 2, 4, and 8), sonelokimab 120 mg augmented load (at weeks 0, 2, 4, 6, 8, and 10), or secukinumab 300 mg (at weeks 0, 1, 2, 3, 4, and 8), with placebo given at weeks 1, 3, 6, and 10 in the sonelokimab 30 mg, 60 mg, and 120 mg normal load groups, at weeks 1 and 3 in the sonelokimab 120 mg augmented load group, and at weeks 6 and 10 in the secukinumab 300 mg group.

During the dose maintenance or escalation period (weeks 12-24), participants assigned to the placebo groupreceived sonelokimab 120 mg (at weeks 12, 14, 16, and then every 4 weeks); those assigned to sonelokimab 30 mg or 60 mg groups with an IGA score of more than 1 were escalated to 120 mg every 4 weeks, and those with an IGA score of 1 or less stayed on the assigned dose at week 12 and then every 4 weeks; those assigned to the sonelokimab 120 mg groups received sonelokimab 120 mg at week 12 and then every 8 weeks (normal load group) or every 4 weeks (augmented load); and those assigned to the secukinumab 300 mg group received secukinumab 300 mg at week 12 and then every 4 weeks. During this period, placebo was given at week 14 in all groups, and at week 16 in the sonelokimab 120 mg normal load group.

In the response assessment with dose-holding period (weeks 24-48), participants in the sonelokimab 30 mg or 60 mg groups who had dose escalation to 120 mg remained on the same regimen regardless of the IGA score at week 24. Participants in the secukinumab 300 mg group also remained on the same regimen regardless of IGA score at week 24. Participants in the sonelokimab 30 mg and 60 mg groups without dose escalation, and all participants in the two sonelokimab 120 mg groups (including placebo rollover participants) were eligible to stop study drug at week 24. Those patients with an IGA score of 0 at week 24 received placebo; these participants resumed the previous dose of sonelokimab every 4 weeks when they had an IGA score of 1 or more (assessed every 4 weeks). Participants in these groups with an IGA score of 1 or more at week 24 continued on the same dosage.

All study treatments were administered as subcutaneous injections. The final dose in all groups was given at week 44, and key efficacy and safety assessments were done at week 52. Efficacy was assessed with IGA (Langley, R G B et al. J Dermatolg Treat 2015; 261:23-31) and PASI (Fredriksson T., et al. Dermatologica 1978; 157:238-244; Weisman S., et al. J Dermatolg Treat 2003; 14:158-165) assessments, which were done by study site personnel at the research sites who were familiar with the scoring systems and specifically trained as part of study conduct.

### Outcomes

The primary outcome was the proportion of participants in the sonelokimab groups with an IGA of clear or almost clear (score 0 or 1) at 12 weeks compared with the placebo group. Secondary outcomes during the placebo-controlled period were PASI responses, change in body surface area of psoriasis, and safety evaluations. A 100% improvement in PASI from baseline (PASI 100 response) is defined as being clinically free of psoriasis. Safety and efficacy were evaluated weekly up to week 4, followed by every 2 weeks until week 16, and then every 4 weeks until week 52. Prespecified exploratory endpoints included dose escalation responses at week 24, the effects of dose-holding after week 24, and the proportion of participants with a Dermatology Life Quality Index (DLQI) score of 0 or 1, assessed every 12 weeks (all prespecified exploratory endpoints are listed in the study protocol). Adverse events reported by participants based on open-ended and non-leading verbal questions by the investigator were collected at each visit.

### Statistical Analysis

Sample size calculations were based on feasibility in the phase 2 setting and assumptions that could enable differentiation between the highest and lowest doses of sonelokimab. No control for multiple comparisons was implemented. The study was not powered for formal comparisons between sonelokimab and secukinumab. The response and safety data for the secukinumab 300 mg group were used for qualitative comparisons.

The primary outcome was analysed in the intention-to-treat (ITT) population; participants with missing data were considered as non-responders (non-responder imputation). The primary active treatment (ie, sonelokimab or secukinumab) comparisons versus placebo were made with the two-sided Cochran-Mantel-Haenszel test, stratified by actual previous biologic use (yes or no) and bodyweight stratum (≤90 kg or >90 kg). Selected sensitivity analyses (missing response imputed with last observation carried forward, using randomised previous biologic use and bodyweight stratum) were done in the ITT population. The safety population was defined as all patients who received the study drug and and was identical to the defined ITT population.

Secondary efficacy and quality of life endpoints were compared between the active treatment groups and the placebo group, and between the sonelokimab groups at each scheduled visit up to week 12, and were compared between treatment groups at week 24 with the CochranMantel-Haenszel test for binary endpoints and an analysis of covariance model, including planned treatment group, the stratum of actual previous biologic use combined with weight as factors, and the baseline value as a covariate in the model for changes from baseline (or percent change from baseline for PASI).

Exploratory analyses of efficacy were done in all participants who were dose-escalated at week 12. At the end of the study, exploratory analyses of efficacy were also done in participants who had active treatment withheld (ie, those with an IGA score of 0 at week 24) and in participants who did not have active treatment withheld (ie, those with IGA score of ≥1 at week 24).

All statistical analyses were done using SAS version 9.4.

A data monitoring committee, which consisted of three dermatologists from institutions that were not involved in the study and who were familiar with clinical studies of biologic agents in psoriasis, met periodically as mandated by a charter put in place at the beginning of the study to review safety information. The committee did not recommend any changes to the study protocol.

### Results

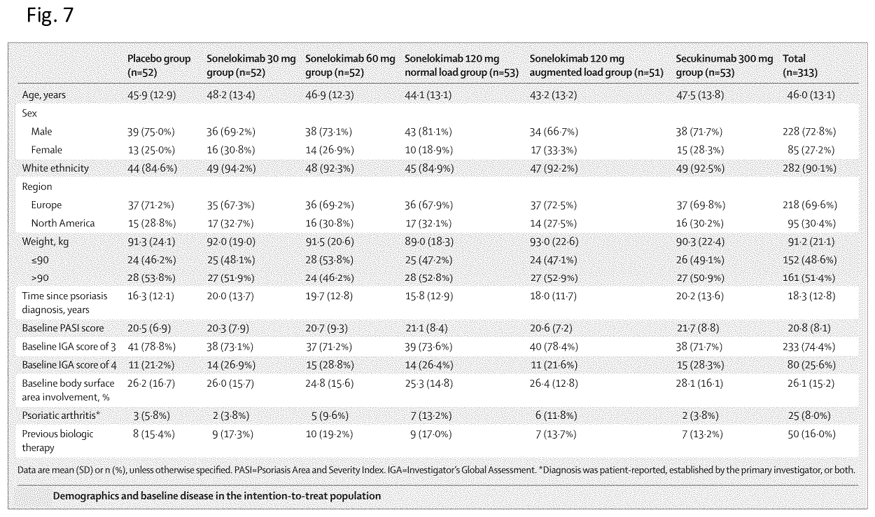

Between Aug. 15, 2018, and Mar. 27, 2019, 383 patients were assessed for eligibility, and 313 were enrolled and randomly assigned to the placebo group (n=52), the sonelokimab 30 mg group (n=52), the sonelokimab 60 mg group (n=52), the sonelokimab 120 mg normal load group (n=53), the sonelokimab 120 mg augmented load group (n=51), or the secukinumab 300 mg group (n=53; see Supplement in Papp et al., Lancet 2021; 397:1564-75). Demographic and baseline characteristics were generally similar between the treatment groups (FIG. 7). The mean age of participants was 46 (SD 13-1), most participants were men (228 [73%] of 313) and white (282 [90%]), the mean duration of psoriasis was 18 years (SD 12.8), and the mean baseline PASI score was 20.8 (8.1).

At week 12, none (0.0% [95% CI 0.0-6.8]) of the 52 participants in the placebo group had an IGA score of 0 or 1 versus 25 (48.1% [34.0-62.4], p<0.0001) of 52 participants in the sonelokimab 30 mg group, 44 (84.6% [71.9-93.1], p<0.0001) of 52 participants in the sonelokimab 60 mg group, 41 (77.4% [63.8-87.7], p<0.0001) of 53 participants in the sonelokimab 120 mg normal load group, 45 (88.2% [76.1-95.6], p<0.0001) of 51 participants in the sonelokimab 120 mg augmented load group, and 41 (77.4% [63.8-87.7], p<0.0001) of 53 participants in the secukinumab 300 mg group.

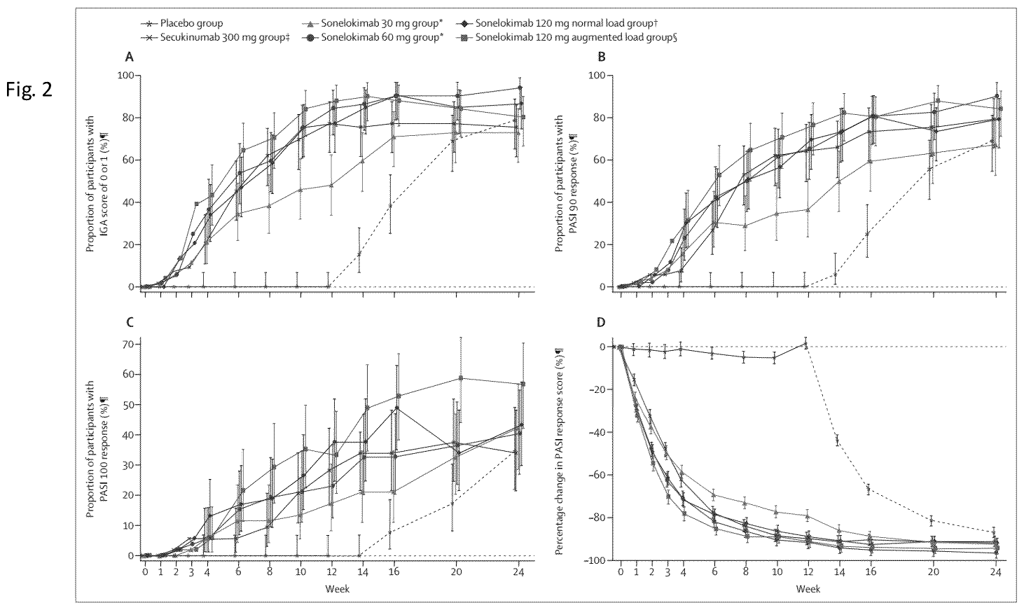

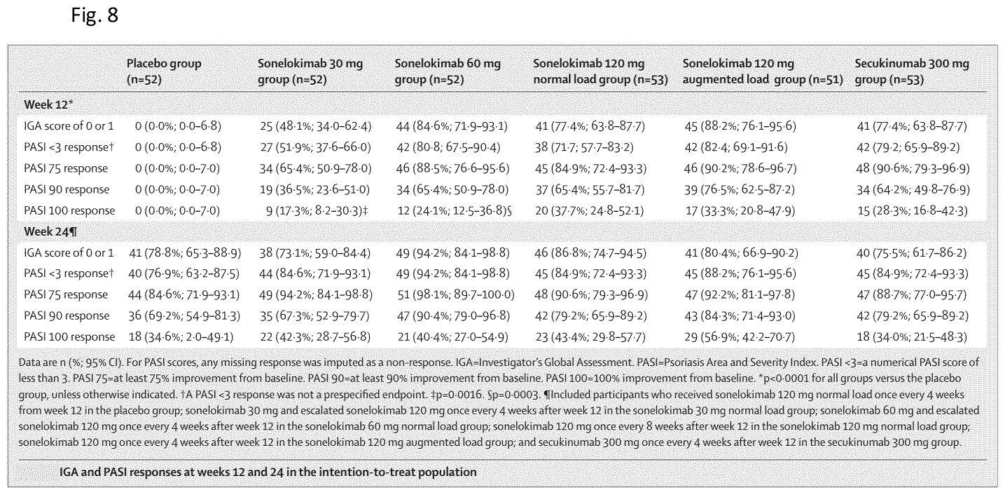

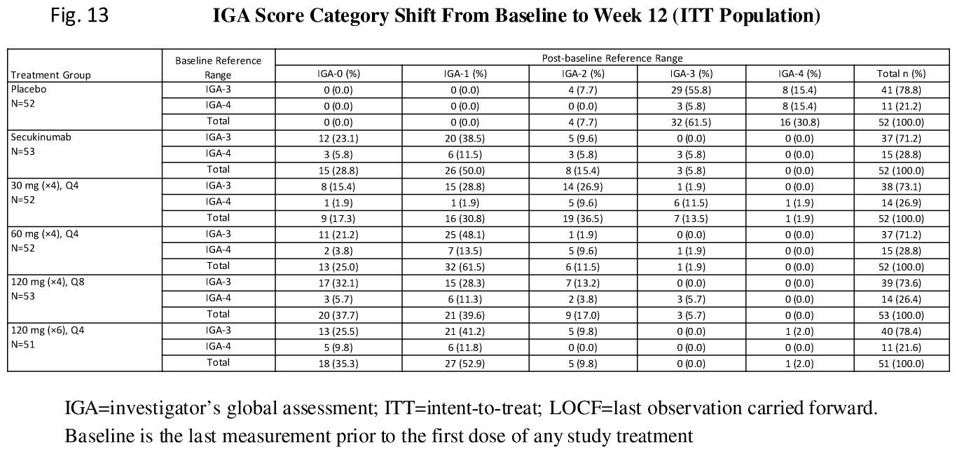

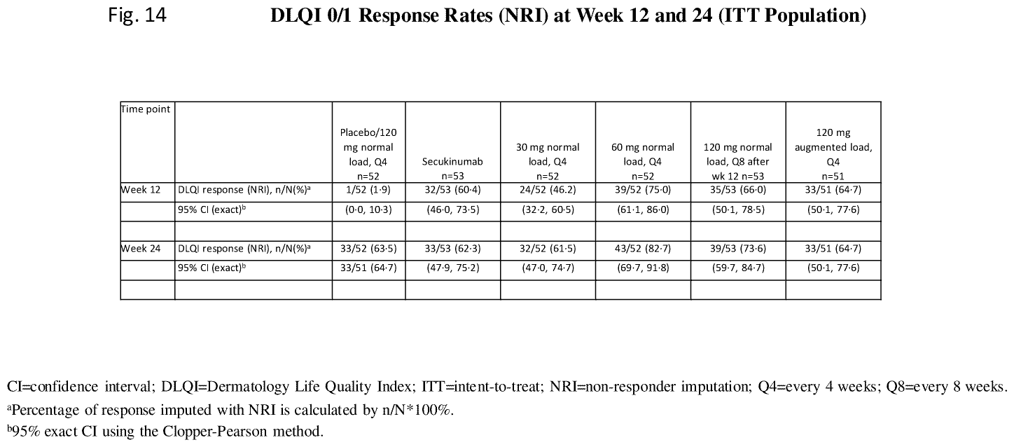

Of the 313 randomised participants, 302 (97%) completed the assigned treatment up to and including week 12 (See Supplement in Papp et al., Lancet 2021; 397:1564-75). Compared with the placebo group, a significantly higher proportion of participants in the sonelokimab 120 mg augmented load group had a PASI 90 response, defined as at least 90% improvement from baseline (39 [76.5%; 95% CI 62.5-87.2] of 51 participants; p<0.0001) and a PASI 100 response (17 [33.3%; 20.8-47.9] of 51 participants; p<0.0001; FIG. 8). A significantly higher proportion of participants in the sonelokimab 120 mg augmented dose group had an IGA of 0 than in the placebo group (FIG. 13). The proportion of participants with an IGA response (ie, an IGA score of 0 or 1) and a PASI 90 response in the active treatment groups began to differ from the placebo group early, with groups receiving higher doses (ie, sonelokimab 60 mg and 120 mg) mostly outperforming the sonelokimab 30 mg group (FIG. 2). Onset of response was rapid, with 16 (31.4%) of 51 participants in the sonelokimab 120 mg augmented load group achieving a PASI 90 response by week 4. No IGA or PASI responses to placebo were observed in the placebo group at any timepoint. Compared with the placebo group, a significantly higher proportion of participants in the secukinumab 300 mg group had a PASI 90 response (34 [64.2%; 49.8-76.9] of 53 participants; p<0.0001) and a PASI 100 response (15 [28.3%; 16.8-42.3] of 53 participants; p<0.0001) at week 12. 131 (63.0%) of all 208 participants on sonelokimab had a DLQI score of 0 or 1 by week 12 compared with only one participant in the placebo group FIG. 14). Changes in the percentage of body surface area involvement with psoriasis between weeks 0 and 12 in participants given sonelokimab were consistent with IGA score and PASI response results (See Supplement in Papp et al., Lancet 2021; 397:1564-75).

Of the originally randomised 313 participants, 297 (95%) completed week 24 (See Supplement in Papp et al., Lancet 2021; 397:1564-75). At week 12, 27 (51.9%) of 52 patients in the sonelokimab 30 mg group and seven (13.5%) of 52 participants in the sonelokimab 60 mg group did not achieve the primary endpoint of an IGA response; these participants had their dosage escalated to sonelokimab 120 mg every 4 weeks. Participants in the sonelokimab 120 mg normal load group received 120 mg at week 12 and every 8 weeks thereafter; patients in the sonelokimab 120 mg augmented load group received 120 mg at week 12 and every 4 weeks thereafter. The proportion of participants in dose-optimised sonelokimab 60 mg and 120 mg groups with an IGA response ranged from 80.4% (95% CI 66.9-90.2) to 94.2% (84.1-98.8); with a PASI 90 response ranged from 79.2% (65.9-89.2) to 90.4% (79.0-96.8); and with a PASI 100 response ranged from 40.4% (27.0-54.9) to 56.9% (42.2-70.7; FIG. 8; FIG. 2 (A-C)). Participants in the placebo group received 120 mg at weeks 12, 14, 16, and every 4 weeks thereafter; at week 24, the proportion of participants with an IGA and PASI response were similar to those observed at week 12 in participants initially given this regimen. In participants who received sonelokimab 120 mg during the first 12 weeks, the proportion of those with an IGA score of 0 or 1, a PASI 90 response, and a PASI 100 response generally peaked by week 16 (FIG. 2 (A-C)). Of note, the proportion of participants on sonelokimab once every 8 weeks in weeks 12-24 with an IGA, a PASI 90, and a PASI 100 response decreased by the time they were due to receive their next dose (ie, by week 20). In all groups, the proportion of participants with a DLQI score of 0 or 1 increased by week 24 compared with week 12 (FIG. 14). At week 24, 40 (75.5%; 61.7-86.2) of 53 participants in the secukinumab 300 mg group had an IGA score of 0 or 1, 42 (79.2%; 65.9-89.2) had a PASI 90 response, and 18 (34.0%; 21.5-48.3) had a PASI 100 response (FIG. 8).

PASI scores improved over time during the placebo controlled period (weeks 0-12) in all active treatment groups, irrespective of dose (FIG. 2 (D)). The mean percentage change from baseline in PASI response scores illustrates the rapid effects of treatment in these participants. During the dose maintenance or escalation period, notable rapid improvements in PASI response scores were observed from week 14 in placebo group participants who were transitioned to sonelokimab 120 mg.

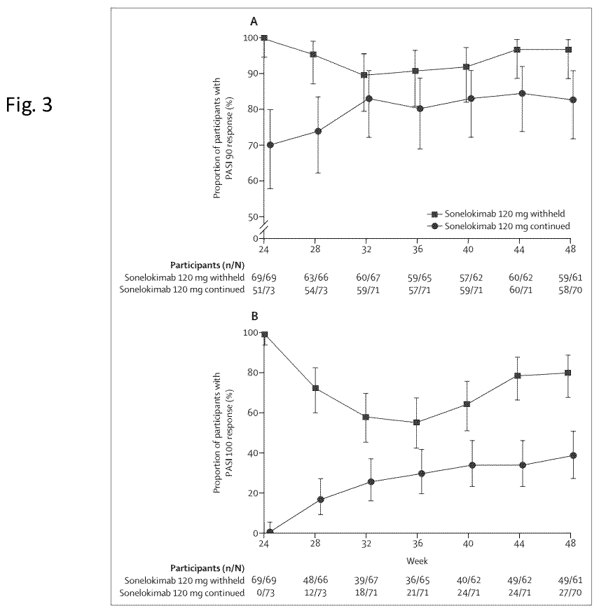

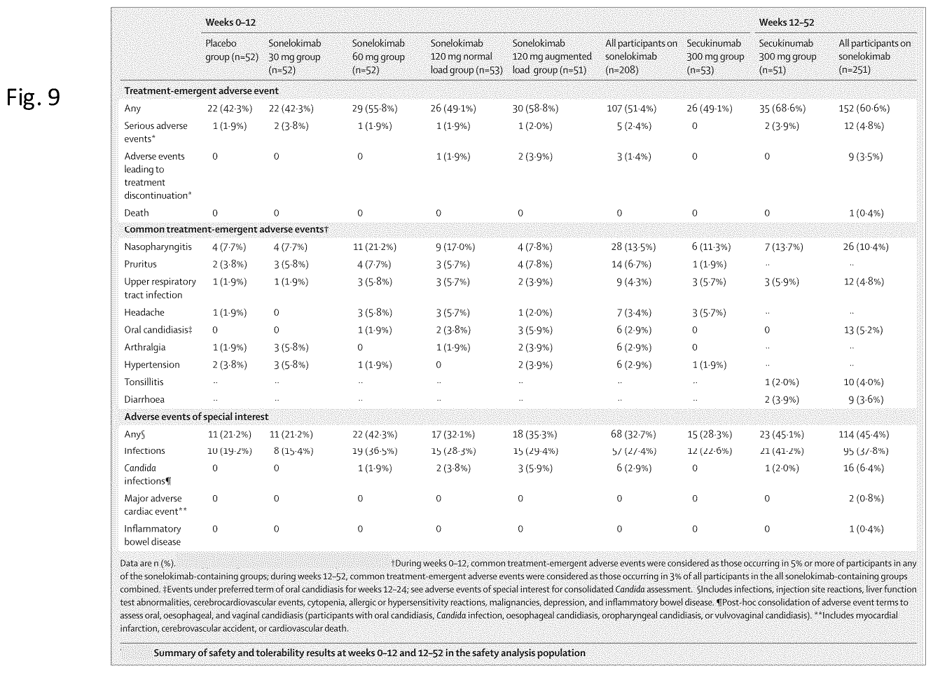

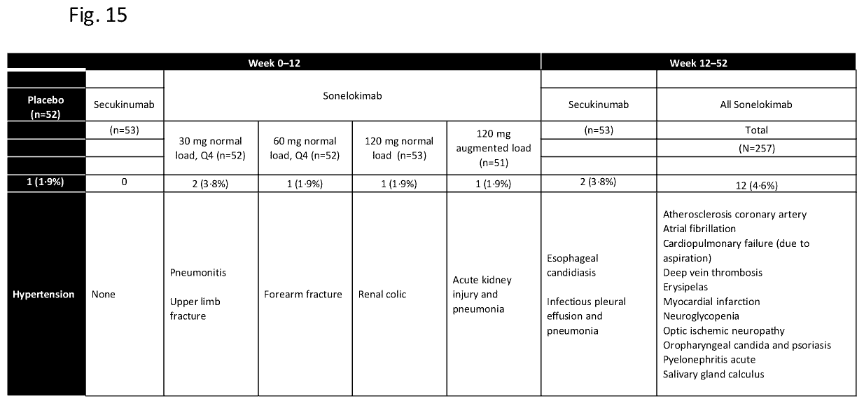

An evaluation of response at 48 weeks with continued administration of sonelokimab was not a goal of the study design. Instead, participants with an IGA score of 0 at week 24 stopped sonelokimab treatment. Interpretation of IGA and PASI response scores for each group are complicated by dose adjustments permitted as per the study protocol (See Supplement in Papp et al., Lancet 2021; 397:1564-75). A post-hoc analysis was done in participants randomly assigned to the placebo group who received sonelokimab 120 mg after week 12 and those randomly assigned to the sonelokimab 120 mg normal load and sonelokimab 120 mg augmented load groups. These participants were combined into a single group of 142 participants, of whom 69 (48.6%) had an IGA of 0 at week 24 and sonelokimab was subsequently withheld. Of these 69 participants, 60 (87.0%) did not maintain an IGA score of 0 over the subsequent 4-12 weeks, but 47 (78.3%) of these 60 participants subsequently re-achieved an IGA of 0 when sonelokimab 120 mg was restarted. Participants who did not have an IGA score of 0 at week 24 continued on sonelokimab 120 mg, and the proportion of those with PASI 90 and PASI 100 responses continued to increase (FIG. 3 (A, B)). Safety was evaluated during the initial placebo-controlled 12-week induction period and the dose-optimisation (secukinumab-controlled) period (ie, the combined dose maintenance or escalation period and response assessment or dose-holding period) between 12 weeks and 52 weeks. After week 12, sonelokimab dosage and exposure varied across treatment groups. Participants on 30 mg and 60 mg sonelokimab with an IGA score of more than 1 at week 12 were given an increased dose. Participants with an IGA score of 0 at week 24 stopped sonelokimab treatment until this response was lost. Participants in the placebo group were only eligible to receive sonelokimab after week 12. Adverse events occurred in 155 (49.5%) of 313 participants, with a slightly greater incidence in sonelokimabcontaining groups (107 (51.4%) of 208) than in the placebo group (22 [42.3%] of 52) at weeks 0-12 (FIG. 9). There was no apparent effect of increasing sonelokimab dose on the incidence of adverse events. During weeks 0-12, the most common adverse events in all participants on sonelokimab were nasopharyngitis (28 [13.5%] of 208 participants), pruritus (14 [6.7%] participants), and upper respiratory tract infections (nine [4.3%] participants). Three participants on sonelokimab 120 mg (one in the normal load group and two in the augmented load group) discontinued treatment because of an adverse event in weeks 0-12: one participant with a pustular rash who did not want to undergo the protocol mandated biopsy, one participant with hypertension, and one participant with acute kidney injury. The participant with acute kidney injury received sonelokimab 120 mg for 1 month, was then prescribed amoxicillin with clavulanic acid and clarithromycin for 3 days for an upper respiratory infection, and was diagnosed with acute tubular necrosis with acute drug-induced nephritis. During weeks 0-12, six participants had a serious adverse event (one in the placebo group, two in the sonelokimab 30 mg group, one in the sonelokimab 60 mg group, one in the sonelokimab 120 mg normal load group, and one in the sonelokimab 120 mg augmented load group; FIG. 9; FIG. 15). No clinically significant signals were identified from the evaluation of laboratory assessments, vital signs, electrocardiograms, or depression and suicidality scales.

During weeks 12-52, participants on sonelokimab were consolidated into one group because of the variation in dosing among groups. Adverse events of special interest were defined based on the known effects of IL-17 modulation. With the possible exception of candidiasis, no dose-response associations with toxicity were apparent. Most Candida infections were easily managed. Four (1.6%) of 251 participants on sonelokimab had absolute neutrophil counts of less than 1000 cells per μL; all cases of neutropenia resolved quickly with no changes to sonelokimab dosing. No other clinically significant signals were identified from the evaluation of laboratory assessments, vital signs, electrocardiograms, or depression and suicidality scales.

One participant on sonelokimab, who was initially given placebo and transitioned to sonelokimab 120 mg at week 12, was admitted to hospital for oral treatment of oropharyngeal candidiasis. One participant in the secukinumab 300 mg group was admitted to hospital for oesophageal candidiasis and received intravenous antifungals. Other notable events were one new diagnosis of Crohn's disease and one death. This Crohn's disease diagnosis was made in one participant receiving sonelokimab 30 mg escalated to 120 mg at week 12, who reported a family history of chronic bowel disease. This participant had intermittent diarrhoea during months 2 and 9 of the study, and had a colonoscopy 11 months into the study. One participant in the sonelokimab 60 mg group died while asleep at home, and was reported to have had a cardiopulmonary failure because of pulmonary aspiration of gastric content.

### Discussion

In this phase 2b study of sonelokimab, doses of up to 120 mg showed rapid and significant clinical benefit when compared with placebo. Participants on the highest dose (120 mg augmented load) showed a rapid response, with 16 (31.4%) of 51 participants achieving a PASI 90 response by week 4, and 39 (76.5%) of 51 participants achieving a PASI 90 response by week 12. Responses were durable. Approximately half (69 [48.6%] of 142) of participants on sonelokimab 120 mg had an IGA score of 0 at week 24. When treatment was withheld in participants with an IGA score of 0 at week 24, modest attrition of response was observed; when treatment was restarted, many (47 [78.3%] of 60) participants re-achieved complete responses. Some differences in responses across different doses and schedules of sonelokimab were apparent and best visualised in the early portions of the mean change in PASI response curves, and in the mid-to-late portions of the PASI 100 response curves. Sonelokimab was generally well tolerated, with a safety profile similar to the secukinumab active control. Overall, a higher incidence of Candida infections was observed in participants on sonelokimab compared with placebo; a single occurrence of oesophageal candidiasis occurred in one participant in the secukinumab 300 mg group. Although the highest dose and schedule of sonelokimab could be used in future clinical studies, additional assessment and modelling could aid in the final selection of the optimal dose and schedule.

The first biologics for the treatment of psoriasis, T-celldirected therapies, were approved 18 years ago. Since then, therapeutic mechanisms have evolved, from tumour necrosis factor (TNF) inhibition, to IL-12 and IL-23 blockade, to IL-17A interference and IL-17 receptor blockers, to IL-23-specific agents, and now there are emerging data on IL-17A/F modulators. Each of these innovations has generated questions about potential increases in efficacy, the benefits of various molecular constructs, and cautions with regards to the need to fully understand potential toxicities. Specifically, with respect to the mechanism of action of IL-17 in psoriasis, IL-17A and IL-17F appear to be the main pro-inflammatory mediators in psoriasis, with IL-17A/A and IL-17F/F homodimers and the IL-17A/F heterodimer as the biologically active molecules. Although secukinumab primarily inhibits IL-17A/A and ixekizumab primarily inhibits IL-17A/A and IL-17A/F, (Eli Lilly and Company. Highlights of prescribing information: TALTZ (ixekizumab) injection, for subcutaneous use. 2020. http://pi.lilly.com/us/taltz-uspi.pdf (accessed Sep. 17, 2020; Paul C., Br J Dermatol 2018; 178: 1003-1005) there is evidence that IL-17F/F also plays a role in the inflammatory cascade. In the presence of TNFα, IL-17F alone is capable of activating pro-inflammatory signalling pathways in human keratinocytes and fibroblasts. (Glatt S., et al. Ann Rheum Dis 2018; 77:523-532; Kolbinger F., et al. J Allergy Clin Immunol 2017; 3:329-32.e8) Furthermore, concurrent IL-17F and IL-17A blockade in vitro decreases synoviocyte and fibroblast mediator production induced by supernatants from T helper 17 cells compared with IL-17A blockade alone. (Glatt S., et al. Ann Rheum Dis 2018; 77:523-532) Brodalumab is a monoclonal antibody targeting IL-17RA that prevents binding by all IL-17 subtypes (Lebwohl M, et al. N Engl J Med 2015; 373:1318-1328) and interferes with both IL-17A and IL-17F.

Brodalumab also blocks other IL-17 family members, including IL-17E (or IL-25), which is downregulated in lesional versus non-lesional psoriasis skin and might have some anti-inflammatory effects. (Monin L., et al. Cold Spring Harb Perspect Biol 2018; 10: a028522; Johnston A., et al. J Immunol 2013; 190:2252-2262) Bimekizumab is a humanised monoclonal antibody that potently and selectively neutralises the biological function of both human IL-17A and IL-17F.7 Clinical phase 2 data on bimekizumab support the notion that IL-17A and IL-17F blockade is effective in the treatment of psoriasis. Papp K A, et al. J Am Acad Dermatol 2018; 79:277-86.e10; Ritchlin C T, et al. Lancet 2020; 8:427-440) The speed and degree of response observed in these phase 2 studies, together with early views of phase 3 data, (Reich K., et al. Lancet 2021; 397:487-498; Gordon K B, et al., Lancet 2021; 397:475-486) support the clinical relevance of IL-17F interference. Our phase 2b study of sonelokimab is the first to include two different IL-17 modulating agents in the same study. Uniquely, sonelokimab is a nanobody that blocks IL-17A, IL-17F, and the IL-17A/F heterodimer. Compared with monoclonal antibodies, the smaller size of sonelokimab might enable differential penetration of the skin and other tissues. The major limitation of this study is the phase 2 scope and the absence of formal comparisons between sonelokimab and secukinumab. Although indications of differences between sonelokimab and secukinumab, such as more rapid effects and higher peak responses, are suggested, this study is not powered to enable confident differentiation. A true comparative approach will require a phase 3 setting, with the optimal dose of sonelokimab prospectively compared head-to-head with antibodies such as secukinumab. The data presented in this report suggest that the addition of IL-17F modulation might provide a fast onset of action, a high ceiling of effect, and possibly more frequent oral fungal infections. Future studies, such as the trial of bimekizumab and secukinumab (NCT03536884) completed in 2020, might contribute to the understanding of IL-17A and IL-17F blockade versus IL-17A blockade alone. In addition, how differences in the exact binding properties of inhibitors of IL-17A/A or IL-17A/F and IL-17F/F, such as bimekizumab and sonelokimab, will influence the benefit-risk profile of these drugs remain to be understood.

### Example 2

### Maintenance Treatment Response

Individual patient-level clinical response data obtained within the highest sonelokimab dose group (120 mg injected every 2 weeks until week 12, every 4 weeks until week 24, and then either withdrawn or continued every 4 weeks; n=51) and the active reference group treated continuously with the IL-17A inhibitor secukinumab (300 mg weekly until week 4 and then every 4 weeks; n=53) were analyzed to gain insight into the “disease-modifying” potential of IL-17A and Il-17F inhibition in psoriasis. See Reich et al., Br J Dermatol. 2022 Apr. 20. doi: 10.1111/bjd.21617, which is incorporated by reference herein in its entirety.

Analyses using the 5-point IGA scale (Langley R G et al., J Dermatolog Treat 2015; 26:23-31) were performed based on the intention-to-treat (ITT) population until week 24 and based on the as observed population thereafter. In the sonelokimab and secukinumab groups, n=4 and n=2 patients discontinued prior to week 24, respectively. Between week 24 and week 48, n=4 patients discontinued in the sonelokimab group (n=2 each lost to follow-up and withdrawal of consent; n=3 patients left the study with IGA=0) and n=2 patients discontinued in the secukinumab group (n=2 withdrawal of consent, both left with IGA=2). Visit intervals between week 24 and week 48 were every 4 weeks. Patients requiring restart of treatment upon disease reoccurrence (IGA≥1) following sonelokimab withdrawal received monthly injections until week 48. Analyses are descriptive post-hoc comparisons with nominal P-values derived from chi-square tests, no corrections for multiple testing were made.

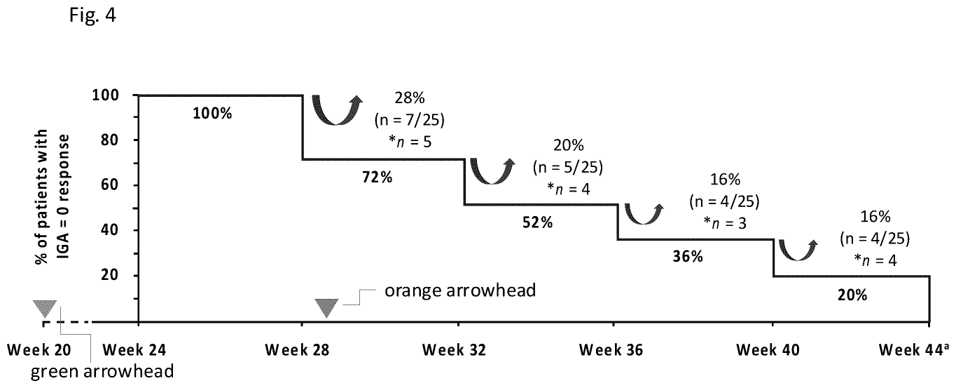

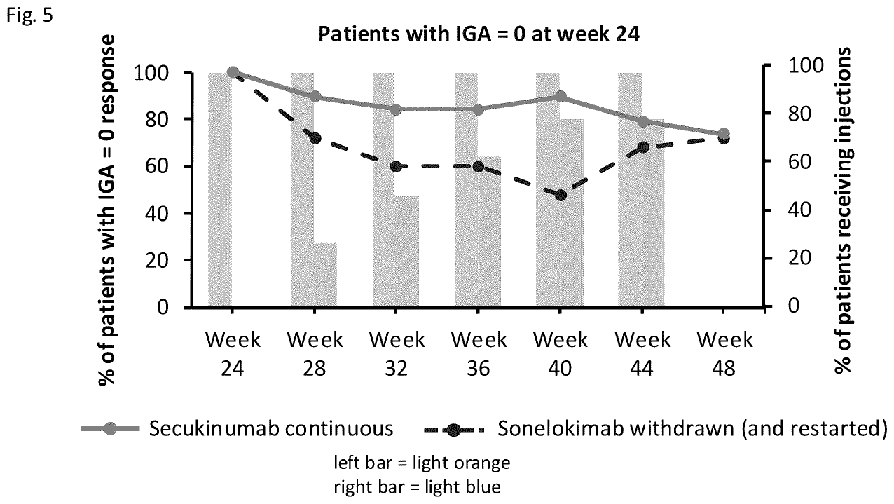

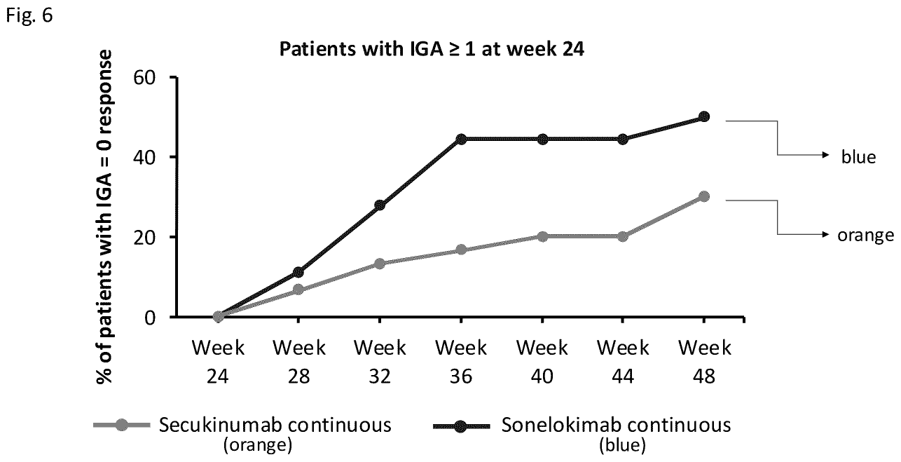

In the sonelokimab 120 mg and secukinumab arms, 56.9% (n=29/51) and 34.0% (n=18/53) of patients achieved complete clearance (Psoriasis area and severity index [PASI]100) at week 24, respectively (ITT-non-responder imputation; P=0.019). Of the 25 patients in the sonelokimab arm followed until week 48 with IGA=0 and treatment withdrawal at week 24, 20% (n=5/25) maintained complete clearance (IGA=0) until week 44 and did not require treatment re-initiation (FIG. 4). Of the remaining 20 patients, n=16/2/2 were retreated with disease activity of IGA=1/2/3, respectively and 80% of retreated patients achieved complete clearance at week 48 (n=16/20). The proportion of patients with complete clearance at week 48 among patients with IGA=0 at week 24 was similar among patients with withdrawal and restart of sonelokimab and patients continuously treated with secukinumab (72.0% and 73.7%, respectively; FIG. 5). Patients in the sonelokimab withdrawal/retreatment group received approximately 50% less total monthly injections between week 24 and week 48 compared with patients in the continuous secukinumab arm. Among patients with active disease at week 24 (IGA≥1), a numerically higher proportion of patients in the sonelokimab arm achieved complete clearance at week 48 compared with the secukinumab arm (FIG. 6).

Analysis and comparison of maintenance of treatment response must consider variability in eligibility for treatment withdrawal (required level of response), treatment duration prior to withdrawal, and therapy half-life. In the Phase 2 study, maintenance of skin clearance (IGA=0) for seven half-lives was observed in approximately 50% of patients following withdrawal from 120 mg sonelokimab once monthly (half-life 12 days; (Svecova et al., J Am Acad Dermatol 2019; 81:196-203) randomized withdrawal at week 24). This result was somewhat higher than the approximately 40% of patients with maintenance of clearance (PASI 100) for seven half-lives following withdrawal of the IL-17A and IL-17F inhibitor bimekizumab (half-life 26 days; randomized withdrawal at week 16) (Committee for Medicinal Products for Human Use (CHMP). Assessment report: Bimzelx (bimekizumab), 24 Jun. 2021. Available at: https://www.ema.europa.eu/en/documents/assessment-report/bimzelx-epar-public-assessment-report_en.pdf (last accessed 24 Nov. 2021; Gordon K B et al., Lancet 2021; 397:475-86). and somewhat lower than the approximately 70% of patients with maintenance of clearance (PASI 100) for seven half-lives following withdrawal of the IL-23 inhibitor risankizumab (half-life 28 days; randomized withdrawal at week 28) (Blauvelt A et al., JAMA Dermatol 2020; 156:649-58; Pang Y et al., Clin Pharmacokinet 2020; 59:311-26). Equivalent data are not available for IL-17A inhibitors; however, the comparable skin clearance rates observed at week 48 between patients continuously treated with secukinumab and those withdrawn from and eventually restarted on sonelokimab may indicate a higher maintenance of treatment response with inhibition of IL-17A and IL-17F, compared with inhibition of IL-17A alone. Further research is warranted to understand the overlap and dissociation of IL-23- and IL-17-driven inflammation in diseases such as psoriasis, the main cellular sources of these cytokines, the mechanisms that underpin “disease modification”, and the differential effects of therapies targeting different molecular members of these pathways.

1. A method of treating an IL-17-dependent condition comprising administering a nanobody which specifically binds to an IL17A/A homodimer, an IL17-A/F heterodimer, and/or an IL-17F/F homodimer to a subject in need thereof at a dose of 30-240 mg every two to four weeks for up to 24 weeks, followed by withdrawal of treatment with the nanobody for responding subjects.

2. The method according to claim 1, in which the administering is performed subcutaneously.

3. The method according to claim 1, wherein the withdrawal of treatment with the nanobody is for a period of 4 weeks or more.

4. The method according to claim 1, which is a method for treating an IL-17-dependent dermatological condition.

5. The method according to claim 4, which is a method for treating psoriasis (including but not limited to plaque psoriasis, moderate-to-severe plaque psoriasis, pustular psoriasis, generalized pustular psoriasis, palmo-plantar psoriasis, scalp psoriasis, guttate psoriasis, erythrodermic psoriasis, inversive psoriasis), atopic dermatitis, discoid lupus erythematosus, alopecia areata, autoimmune urticaria, bullous pemphigoid, dermatitis herpetiformis, hidradenitis suppurativa, linear IgA dermatosis, morphea, pemphigus vulgaris, or pyoderma gangrenosum.

6. The method according to claim 1, which is a method for treating psoriatic arthritis, axial spondyloarthritis including ankylosing spondylitis, systemic lupus erythematosus, rheumatoid arthritis, vasculitis, Sjogren's syndrome, juvenile idiopathic arthritis, granulomatosis, Behcet's disease, antiphospholipid syndrome, giant cell arteritis, scleroderma, polyarteritis nodosa, or Takayasu disease.

7. The method according to claim 1, which comprises administering the nanobody at a dose of 30, 60, 120, or 240 mg every two to four weeks for up to 24 weeks.

8. The method according to claim 1, wherein the dose is increased between week 2 and week 24 if the subject has an IGA score of more than 1.

9. The method according to claim 1, wherein the nanobody comprises sonelokimab.

10. The method according to claim 1, which comprises a method for treating psoriasis and which achieves full skin clearance in the subject by as early as week 4 to week 8.

11. A method of disease modification comprising administering 30-240 mg of a nanobody to a subject suffering from IL-17-dependent condition every two weeks, wherein
the nanobody specifically binds to an IL17A/A homodimer, an IL17-A/F heterodimer, and/or an IL-17F/F homodimer; and
the treatment is for a period of no less than 4 weeks.

12. The method according to claim 11, which comprises administering the nanobody every two weeks until week 12, and then every four weeks until week 24.

13. The method according to claim 11, in which administration of the nanobody is paused or permanently stopped at week 24.

14. The method according to claim 11, which achieves normalization of peripheral IL-17A, IL-17F, CCL20, CXC11, DEFB4A, CXCL8, LCN2, CAMP, KRT16, IL-13, IL-23, IL-31 and/or IL-22 in the subject by no later than week 24.

15. The method according to claim 11, which achieves normalization of cutaneous IL-17A, IL-17F, CCL20, CXC11, DEFB4A, CXCL8, LCN2, CAMP, KRT16, IL-13, IL-23, IL-31 and/or IL-22 in the subject by no later than week 24.

16. The method according to claim 14, in which the normalization is sustained for at least 4, 8, 12, 16, and/or 20 additional weeks after treatment has been paused or stopped.

17. The method according to claim 11, which comprises:
administering the nanobody for a total of at least 4 weeks and preferably for a total of no more than 24 weeks,
pausing the treatment for a period of more than two weeks, and then
re-initiating treatment if one or more symptoms of the IL-17-dependent condition recur.

18. The method according to claim 17, in which administration is re-started when the subject has an IGA score of 1 or more.

19. The method according to claim 17, wherein re-initiating treatment comprises administering 30-240 mg of the nanobody every two weeks.

20. The method according to claim 17, wherein the IL-17-dependent condition is a dermatological condition.

21. The method according to claim 20, which is a method for treating psoriasis (including but not limited to plaque psoriasis, moderate-to-severe plaque psoriasis, pustular psoriasis, generalized pustular psoriasis, palmo-plantar psoriasis, scalp psoriasis, guttate psoriasis, erythrodermic psoriasis, inversive psoriasis), atopic dermatitis, discoid lupus erythematosus, alopecia areata, autoimmune urticaria, bullous pemphigoid, dermatitis herpetiformis, hidradenitis suppurativa, linear IgA dermatosis, morphea, pemphigus vulgaris, or pyoderma gangrenosum.

22. The method according to claim 21, wherein the nanobody comprises sonelokimab.

23. The method according to claim 21, in which the administering is performed subcutaneously.

24. A method of treating an IL-17-dependent dermatological condition comprising administering a nanobody which specifically binds to an IL17A/A homodimer, an IL17-A/F heterodimer, and/or an IL-17F/F homodimer to a subject in need thereof at a dose of 30-240 mg every two to four weeks continuously for more than 24 weeks to achieve full skin clearance.

25. The method according to claim 24, which achieves a complete clearance of the disease in 40% or more of subjects still exhibiting symptoms of the IL-17-dependent dermatological condition at week 24 of treatment.

26.-40. (canceled)
